(19) United States
(12) Patent Application Publication
(10) Pub. No.: US 2005/0276456 A1
(43) Pub. Date: Dec. 15, 2005

(54) Cell-operating device

(76) Inventors: Toshiyuki Yamato, Kadoma-shi (JP); Yoshitaka Matsumoto, Kyoto-shi (JP); Katsuaki Hayashi, Osaka-shi (JP); Isamu Oh, Sennan-gun (JP); Setsuya Sato, Takatsuki-shi (JP)

Correspondence Address: HENNEMAN & SAUNDERS, 714 WEST MICHIGAN AVENUE, THREE RIVERS, MI, 49093, US

(21) Appl. No.: 11/139,015

(22) Filed: May 26, 2005

(30) Foreign Application Priority Data
May 27, 2004 (JP) 2004-157874
Nov. 29, 2004 (JP) 2004-344603

(51) Int. Cl. G06K 9/00; C12M 1/34

(52) U.S. Cl. 382/128; 435/287.1

(57) ABSTRACT

The present invention provides a cell-operating device comprising a laser light source and a microscope to observe cell sample fluorescently labeled and a camera to take the images of the cell sample that is observed with said microscope and a computer to capture the digital image data obtained by said camera and a group of mirrors comprising electronic control mirrors of which optical axis can be adjusted so that the laser light irradiated from said laser light source can be guided in to said microscope. The computer includes a binarization program for binarizing said digital image data by identifying whether each pixel of said digital image data captures a part of said cell sample according to the fluorescence luminance of each pixel. The computer has an identification program for identifying the boundaries between adjacent different values in the binarized digital image and joins the boundaries into an isolated closed curve, and a data processing program for processing said binarized digital data so that in all the area within said isolated closed curve a cell exists, and a calculation program to divide said binarized digital image data, after running said data processing program, into sections with a predefined size and identify the center of gravity coordinate of each section, and an adjustment program to adjust the optical axis of said electronic control mirrors so that said laser irradiates said center of gravity coordinate. Thus, the present invention is a cell-operating device that the laser light from said laser light source irradiates said center of gravity coordinate.

### RELATED APPLICATIONS

This application claims the right of foreign priority to Application No. Tokugan 2004-157874, filed in Japan on May 27, 2004, and to Application No. Tokugan 2004-344603, filed in Japan on Nov. 29, 2004, by the same inventors, both of which are incorporated herein by reference in their entirety.

### BACKGROUND OF THE INVENTION

1. Technical Field

The present invention relates to a cell-operating device and cell-operating method, more particularly, relates to a cell-operating device and cell-operating method that accurately destroy specific unnecessary cells and, arrange specific necessary cells for a short period of time.

2. Description of the Background Art

In general, a cell-operating process for cultivating cells and forming novel cells requires a high accuracy of choosing cells and a prompt shift-action of cells. Taking an example of a low accuracy of choosing cells, it results in the growth and fusion of unnecessary cells. Besides, if a shift-action of cells is not promptly occurred, targeted cells for an operation lose their freshness.

For example, cell treatments require an accuracy of choosing more specific cells. With respect to cell treatments, treatments for hematopoietic organ tumors such as malignant lymphoma and leukemia are used as a high-dose consolidation therapy and radiation exposure. Also, a method for transplanting hematopoietic stem cells is used in order to treat myelopathy caused by the tumor's side effect.

Other than those above, with respect to cartilage defects, cartilage cell treatments for implanting cartilage cells, as well as for neurodegenerative diseases cell-stem treatments such as nerve cell treatments for implanting nerve cells, have been used. As for these cell-stem treatments, however, undesired cells must be completely removed from implanted cells prior to the transplantation.

Taking an example of hematopoietic stem cell treatments for the above-mentioned hematopoetic organ tumors, it is considered as a favorable way, from the standpoint of prevention against graft-versus-host diseases, that patients' stem cells are ingested and cultivated, and those cultivated stem cells are implanted. However, there is a possibility to contain cells including cancer cells in the ingested stem cells, which might recur hematopoetic organ tumors if implanted without removing cancer cells.

Japan Patent Publication 2003-529340 discloses a method of destroying cells by labeling unnecessary cells and irradiating those labeled cells with a laser.

As the representative example of cell-shift operations, pearl-chain formation is most known, which forms novel cells by trapping specific cells with a laser, shifting those trapped cells to an appointed place, and fusing several those shifted cells. Thus, a well-known method for forming a pearl-chain is that an alternating voltage is applied to electrodes, providing a suspension containing several cells for the facing electrodes. With this method, the pearl-chain can be formed so that cells are arranged in linear groups and parallel to the electric field by adjoining near other cells.

Japan Patent Publication H07-31455 discloses a method of peal chain formation. Thus, the method uses the technique that applies an alternating voltage after shifting specific cells adjacent to electrodes with a laser light.

### SUMMARY OF INVENTION

In order to destroy cells with laser irradiation, the nucleus must be destroyed by accurately irradiating itself with a laser beam. Although the region where cells are irradiated with a laser beam (hereinafter referred as “spot”) is constant, the cell size varies. Therefore, as the cell size became bigger, probability that a laser beam hit the nucleus decreased, which brought about a lower rate of destroying cells. Besides, unlike plant cells, human cells are covered with a soft cell membrane (not cell walls). Since human cells have a variety of forms, laser beam did not always hit the nucleus even if its center was irradiated with a laser beam. Therefore, there was no method for accurately destroying unnecessary cells by conventional technology.

Furthermore, conventional technology has a problem of treating necessary cells. Thus, the technology disclosed in Japan Patent Publication H07-31455 has a great difficulty in controlling forms and sizes of arranged cell groups. Thus, the technology incurs a great deal of time and effort. Besides, when it takes a long time to control the size and form, cells adhere to the surface of the well on which a suspension is placed, which makes it more difficult to shift cells by laser-trapping.

Therefore, considering the above-mentioned problems, a primary object of the present invention is to provide a cell-operating device and cell-operating method in which unnecessary cells are accurately destroyed as well as necessary cells are shifted with high controllability.

### ADVANTAGEOUS EFFECTS OF THE INVENTION

The present invention according to the claim 1 is capable of accurately destroying cells since the invention can irradiate almost every region of a cell with a laser beam regardless of cell size and its form.

The present invention according to the claim 2 prevents unnecessary parts from being irradiated with a laser beam. Thus, it prevents cells that do not need to be destroyed from being damaged by the beam.

The present invention according to the claim 3 minimizes a laser beam that irradiates and overflows a contour of a cell, as well as it further reduces the risk of damaging cells that do not need to be destroyed. In addition, the present invention is capable of effectively and accurately destroying cells since the entire cell can be accurately irradiated with a laser beam and sectional numbers are also minimized so as to irradiate the entire cell with a leaser beam.

The present invention according to the claim 4 facilitates the observation of cells.

The present invention according to the claim 5 is capable of destroying one's desired cells by a mouse-device operation.

The present invention according to the claim 6 is capable of destroying cells within one's desired region.

The present invention according to the claim 7 is capable of separating between viable and dead cells.

The present invention according to the claim 8 provides a suspension only from viable cells for the well.

The present invention according to the claim 9 separates cells from the surface of the well by an alternating voltage. Besides, the present invention is capable of shifting cells for a short period of time due to a condition in which cells are detached on the surface.

The present invention according to the claim 10 is capable of fixing cells in a groove.

The present invention according to the claim 11 facilitates the operation for selecting the region of an image and of a cell-operation.

### BRIEF DESCRIPTION OF DRAWINGS

The present invention is described with reference to the following drawings, wherein like reference numbers denote substantially similar elements:

FIG. 1 shows a schematic view indicating the cell-operating device according to the present invention.

FIG. 2 shows a binarization process of the method for destroying cells according to the present invention. FIG. 2(a) indicates the image of cells scanned into a computer by a camera. FIG. 2(b) represents the first stage of the binarization process. FIG. 2(c) represents the second stage of the binarization process. FIG. 2(d) represents the third stage of the binarization process.

FIG. 3 shows an example of size setting for sectional regions according to the present invention.

FIG. 4 shows a flow chart indicating a series of processes of the method for destroying cells according to the present invention.

FIG. 5 shows an overhead view of the well according to the present invention.

FIG. 6 shows an example of an operation using a display.

FIG. 7 shows an example of an operation using a display.

FIG. 8 shows one embodiment of the well provided with the separation-recollecting function.

FIG. 9 shows the well with electrodes.

FIG. 10 shows one process of a cell-shift operation.

FIG. 11 shows one process of a cell-shift operation.

FIG. 12 shows one process of a cell-shift operation.

FIG. 13 shows one process of a cell-shift operation.

FIG. 14 shows embodiments of the well with grooves.

FIG. 15 shows one process of a cell-shift operation using the well with a groove.

FIG. 16 shows one process of a cell-shift operation using the well with a groove.

FIG. 17 shows one process of a cell-shift operation using the well with a groove.

FIG. 18 shows cell arrangements obtained by the well with a groove.

FIG. 19 shows an alternative well with a groove.

FIG. 20 shows an alternative well with electrodes.

FIG. 21 shows embodiments for shifting cells adhering to the bottom of the well.

### DESCRIPTION OF THE PREFERRED EMBODIMENTS

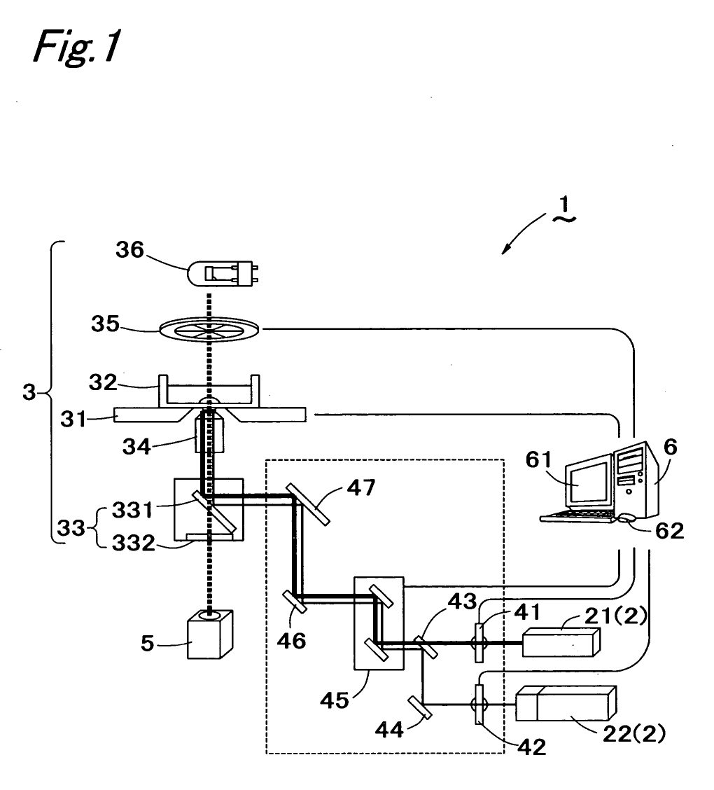

Hereinafter, preferred embodiments of the cell-operating device and the method fro operating the cells according to the present invention are described with referring to the drawings. FIG. 1 is a schematic view of the cell-operating device according to the present invention.

A cell-operating device (1) is composed of a laser light source (2), a microscope (3), a group of mirrors (4) comprising plurality of mirrors that reflects the laser light radiated from the laser light source (2) to guide the laser light into the microscope (3), a imaging device (camera) (5) that takes images of the cells that are magnified by the microscope (2), and a computer (6) that processes the image data obtained by the imaging device (5).

In the example of FIG. 1, the laser light source (2) consists of a first laser light source (21) that beams IR laser and a second laser light source (22) that beams UV laser.

The IR laser beamed from the first laser light source (21) is used as a trapping laser for trapping and operating cells. For example, YAG laser (1060 nm), Nd:YLF laser (1047 nm) and DPSS laser (1064 nm) can be used for the IR laser. However usable IR lasers are not limited to these IR lasers. Any IR laser can be used as long as the laser allows operations such as moving cells without damaging the cells.

Any types of the UV laser capable of destroying cells can be beamed from the second laser light source (22). Such UV laser includes pulse laser, such as Nd: YAG laser, femtosecond titanium saphire laser, femtosecond fiber laser, nanosecond YAG laser, nano/pico second VYO₄ laser and excimer laser.

A group of mirrors (4) consists of electric shutters (41, 42) incorporated directly after the first light source (21) and the second light source (22), dichroic mirrors (43, 44) respectively incorporated in the downstream of the electric shutters (41, 42), an electric mirror unit (45) that the laser light from the dichroic mirrors (43, 44) is sent to, a dichroic mirror (46) incorporated in the downstream of the electric mirror unit (45), and a dichroic mirror (47) that is incorporated in the downstream of the electric mirror unit (46) and sends the light to the microscope (3).

The electric shutters (41, 42) are respectively deployed in front of exit aperture of the first laser light source (21) and the second laser light source (22).

The electric shutters (41, 42) are enabled to open or close separately based on the control signal sent from the computer (6). This enables to send laser light beamed from the first laser light source (21) and the laser light beamed from the second light source (22) to the microscope (3) selectively.

The dichroic mirror (43) is deployed in front of the electric shutter (41). The dichroic mirror (44) is deployed in front of the electric shutter (42).

The laser light beamed from the first laser light source (21) straightly reachs the dichroic mirror (43) via the electric shutter (41).

The laser light that is beamed from the second laser light source (22) passes the electric shutter (42). Then the direction of the laser light is altered by the dichroic mirror (44) so that the light goes to the dichroic mirror (43). The dichroic mirror (43) sends the laser light from the first laser light source (21) directly to the mirror unit (45) while the dichroic mirror (43) changes the direction of the laser light from the second laser light source (22) to send the laser light to the electric mirror unit (45). Beyond the dichroic mirror (43), the laser light from the first laser light source (21) and the laser light from the second laser light source (22) follow the same path.

After passing the dichroic mirror (43), the laser light reaches the electric mirror unit (45). The electric mirror unit (45) has two electronic control mirrors that are controlled separately based on the control signals sent from the computer (6). There are two directions in operating the laser light on the well (32) placed on the electric stage (31) of the microscope (3). One of the electronic control mirror controls the laser light scanning in X direction and the other electronic control mirror controls the laser light scanning in Y direction.

The electric mirrors such as galvanometer mirrors and mirrors driven by a piezo actuator and the like can be used.

After passing the electric mirror unit (45), the laser light reaches the dichroic mirror (46). The dichroic mirror (46) changes the direction of the laser light. After the laser light passes the dichroic mirror (46), the direction of the laser light is changed by the dichroic mirror (47). The laser light is then introduced into the microscope (3).

The microscope (3) consists of a mirror unit (33) that receives the laser light from the dichroic mirror (47), an object glass (34) deployed above the electric stage (31), a well (32) placed on the electric stage (31), an electric shutter (35) deployed above the well (32) and the light source (36) deployed above the electric shutter (35).

The mirror unit (33) has a dichroic mirror (331) and an absorption filter (332).

The dichroic mirror (47) sends the laser light to the dichroic mirror (331). The dichroic mirror (331) changes the direction of the laser light and sends upwardly the laser light to the object glass (34).

The object glass (34) collects the laser light sent from the dichroic mirror (47) and sends the collected light into the well.

The light source (36) irradiates the well (1) with light. The electric shutter (35) is incorporated between the light source (36) and the well (32). The electric shutter (35) adjusts the amount of light sent from the light source (36) to the well (32).

After passing the well (32), the light passes the object glass (34) and reaches the mirror unit (33). The dichroic mirror (331) of the mirror unit (33) passes the light from the light source (36) downwardly. After passing the dichroic mirror (331), the light reaches the absorption filter (332). The absorption filter (332) selectively passes the visible light component of the light irradiated from the light source (36).

After passing the absorption filter (332), the visible light component reaches the camera (5) disposed below the absorption filter (332).

As the camera (5), any cameras, such as CCD camera, CMOS camera and the like can be used. Such camera (5) digitalizes the images sent thereto.

Images of the well (32) taken by the camera (5) are sent to the computer (6) and undergoes the image processing described below.

The electric stage (31) can move in two directions, namely X direction and Y direction, on a horizontal plane. This movement is based on the control signals from the computer (6).

The well (32) accommodates the cell sample labeled for identifying the cells to be destroyed. In this embodiment, targeted cells are cancer cells. However, the targeted cells of the present invention are not limited to these.

To label the cells, labeled antibody is introduced into the cell sample. The labeled antibody has an affinity for the substance that occurs on the surface of a cancer cell. Such labeled antibody includes antibodies such as anti-integrin antibody, anti-CD 44 antibody, anti-MUC-1 antibody, anti-cytokeratin antibody, anti-epidermal growth factor antibody, anti-insulin-like growth factor antibody, and anti-insulin-like growth factor receptor antibody and a part of each antibody that includes antigen recognition region. Such antibodies also includes polypeptide such as collagen, oligopeptide, oligopeptide having RGD sequence, glycoprotein such as fibronectin, polysaccharide such as hyaluronic acid and phosphomannan, oligosaccharide such as mannose 6-phosphate pentamer, monosaccharide such as mannose 6-phospate, vitamin acid such as retinoic acid. It is preferable to use anti-insulin-like growth factor antibody or mannose 6-phosphate pentamer since these antibodies do not interact with normal cells such as leucocyte and blood erythrocyte.

The labeled antibodies selectively attach to cancer cells and cover the surface of the cells.

To the covered cells, enzyme and fluorescence reagent are added. Such enzyme includes, for example, peroxidase, arylphosphatase and such fluorescence reagent includes, for example, fluorescein-iso-thiocyanate, tetramethylrodamin isothiocyanate and so on. Added fluorescence reagent attaches to the labeled antibodies. As a result, cancer cells become fluorescently labled.

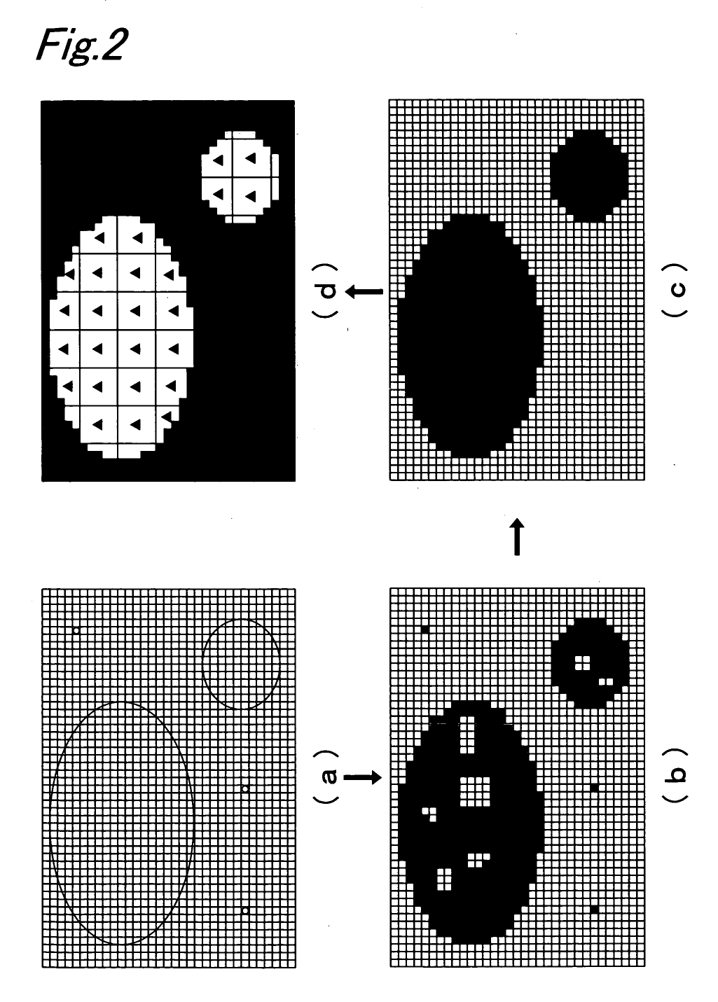

FIG. 2 shows the binarization process in the method for operating the cells according to the present invention. FIG. 2 (a) represents the image of the cells introduced to the computer (6) via camera (5). FIG. 2 (b) represents the first step of the binarization process. FIG. 2 (c) represents the second step of the binarization process. FIG. 2 (d) represents the third step of the binarization process.

When cell samples are labeled as described above, only the targeted cells (cancer cells in the present embodiment) become fluorescent. Thus the computer (6) captures the data for the image exclusively of fluorescent cancer cells. (FIG. 2 (a))

The images are meshed on a pixel by pixel basis. The value “0” is assigned to a mesh that has luminance below the predefined threshold value while the value “1” is assigned to a mesh that has luminance above the predefined threshold value (FIG. 2 (b)). Note that in FIG. 2 the meshes having the value “0” is painted black and the meshes having the value “1” is circled.

After assigned a value of either “1” or “0” as shown in FIG. 2 (b), a boundary between a “1” mesh and a “0” mesh is identified. Each boundary is joined into an isolated closed curve. If the labeled antibody or the fluorescence reagent attached to the labeled antibody in the process of labeling is not sufficient, another closed curve may be created in a closed curve. In this case, the outmost closed curve is identified as the isolated closed curve.

After the isolated closed curve is identified, the value “1” is assigned to the meshes within the isolated closed curve (FIG. 2 (c)). Then the number of meshes haivng the value “1” is counted to calculate the square measure of the area within the isolated closed curve. If the area within the isolated closed curve is smaller than the set value, the laser is not irradiated in the area. This prevents the laser from irradiating the outside of the targeted cells.

Next, each value of “0” and “1” shown in FIG. 2 (c) is inverted (in FIG. 2 (d) the colors black and white are reversed). Then image data is divided into sections with a predefined size.

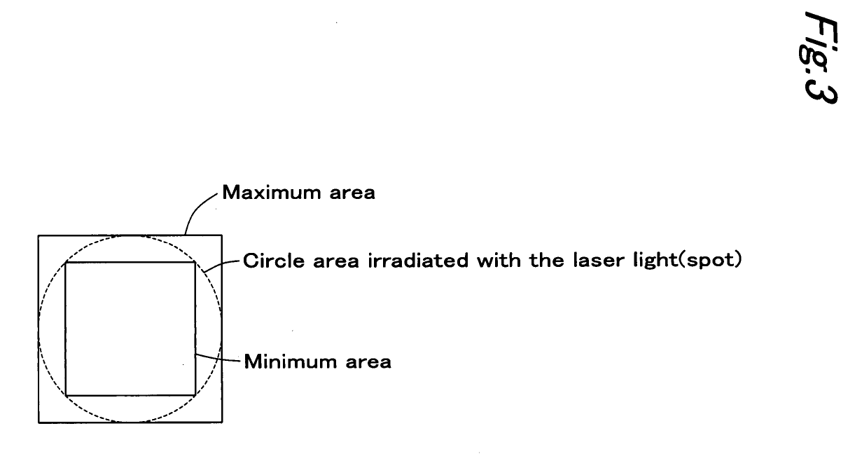

FIG. 3 shows an example of the size of a section.

It is preferable to define the size of the rectangular area between a minimum size and a maximum size. As shown in FIG. 3, a section with the minimum size is the rectangular area that the circle area irradiated with the laser light (i.e. spot) circumscribes. On the other hand, the section with maximum size is the rectangular area that a circle area irradiated with the laser light inscribes. If the size of the sections is below this range, the cell destruction rate is likely to fall. If the size of the sections is above this range, the laser light that irradiates the outside of the cells increases. Thus the risk of damaging the cell that need not be killed increases.

The number of the pixels having the value “0” within every section is counted. Both the square measure and the center of gravity coordinate of each section in which cells exist are calculated. (in FIG. 2, each center of gravity is indicated with a triangular mark). If some sections are smaller than the predefined size, the laser does not have to be irradiated therein.

After the center of gravity coordinate is calculated, laser light is irradiated from the second laser light source (22). The computer (6) adjusts the optical axis of each electronic control mirror incorporated in the electric mirror unit (45) according to the calculated center of gravity coordinate. Irradiating place of the laser light is controlled by the electronic control mirror. As a result, the laser light is directed to the center of gravity coordinate described above.

Since the irradiation spot of the laser light is defined as described above, the laser light can provide light energy to the cells of any shape and largeness and surely kill the cells. In addition, as the laser light irradiates the center of gravity of each section, the amount of the laser light that irradiates the outside of the targeted cells is small. This suppresses the damage to the non-targeted cells around the targeted cells.

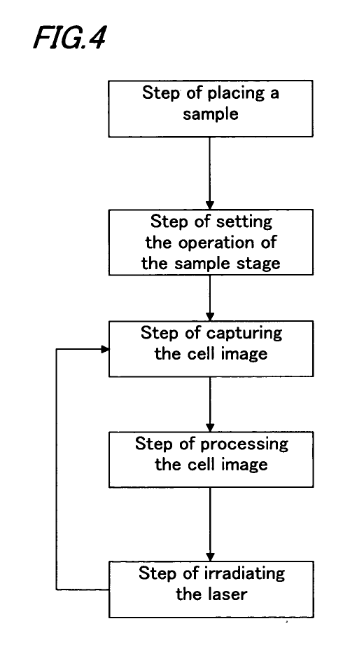

FIG. 4 is a flow chart that shows a series of steps of the method for destroying the cells according to the present invention.

The method includes a step of placing a sample, a step of setting the operation of the sample stage, a step of capturing the cell image, a step of processing the cell image and a step of irradiating the laser.

First, in a step of placing a sample, a translucent white strip is placed on the electric stage (31) as a sample for correcting the coordinate. By using this strip, the coordinate of the image data is adjusted to correspond to the laser-irradiating spot. A value for a parameter used in controlling the optical axis of the electric mirror unit (45) is input into the computer (6). Then laser light is directed to the sample and an image of the sample in this state is taken by the camera (5). The obtained image has a laser spot on the sample. Computer (6) calculates the coordinate of the center of the spot. By comparing the coordinates of the center of the image and the center of the spot, a correction value for correcting the coordinate is calculated. Thus the coordinates of laser irradiating spot and that of image processing are homologized.

The well (32) accommodating a cell sample that has undergone labeling process described above is placed on the electric stage (31).

Then the operation of the electric stage (31) is defined in the step of setting the operation of the sample stage.

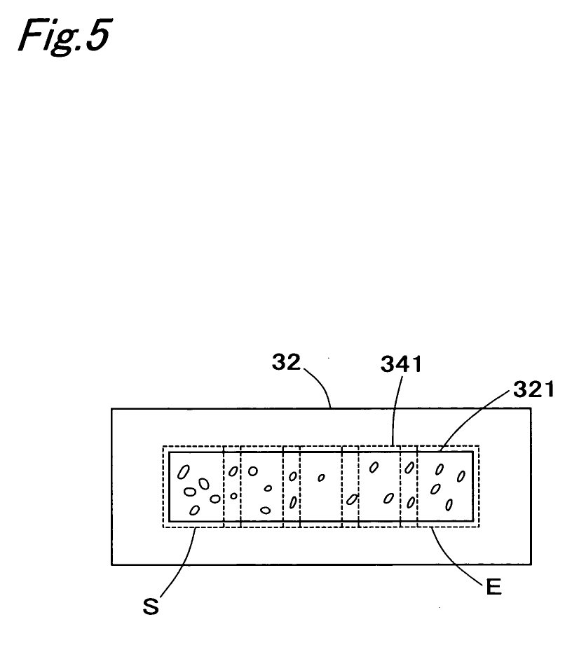

FIG. 5 is a top view of the well (32). Note that the dotted areas in FIG. 5 are the observable areas (341) through the object glass (34).

First, one end of a sample housing (321) that accommodates the well having a sample is set as a starting area (S) and the other end is set as an end area (E). Then the feed pitch of the electric stage (32) is set between the starting area (S) and the end area (E). It is preferable to make an overlap between adjacent observable areas. This enables to observe all samples accommodated in the well (32).

After that, in the step of capturing the cell image, the digital image of the observable areas (341) through the object glass (34) is taken by the camera (5). Then the image data is sent to the computer (6).

In the step of processing the cell image, the image data undergoes the binariztion process as described above. In this step, the image data is also divided into the sections and the center of gravity coordinate of each section is calculated.

Next, in the laser irradiating step, the optical axis of each electronic control mirror incorporated in the electric mirror unit (45) is adjusted. The adjustment is carried out so that the laser light from the second laser light source (22) is directed to the calculated center of gravity coordinate. After all the centers of gravity coordinates in an observable area (341) of the object glass (34) are irradiated with the laser light, the electric stage (32) moves one by one so that the object glass (34) can observe the adjacent observable area (341) in order to allow the area to undergo the series of steps from the cell image capturing steps to laser irradiating steps. This ensures that all the cells on the well are destroyed by the laser light.

The settings for the laser light, such as irradiating duration, power and pulse frequency may be varied according to the type of the targeted cells.

Described above is merely a basic embodiment relating to cell destruction and many variations of this embodiment are possible.

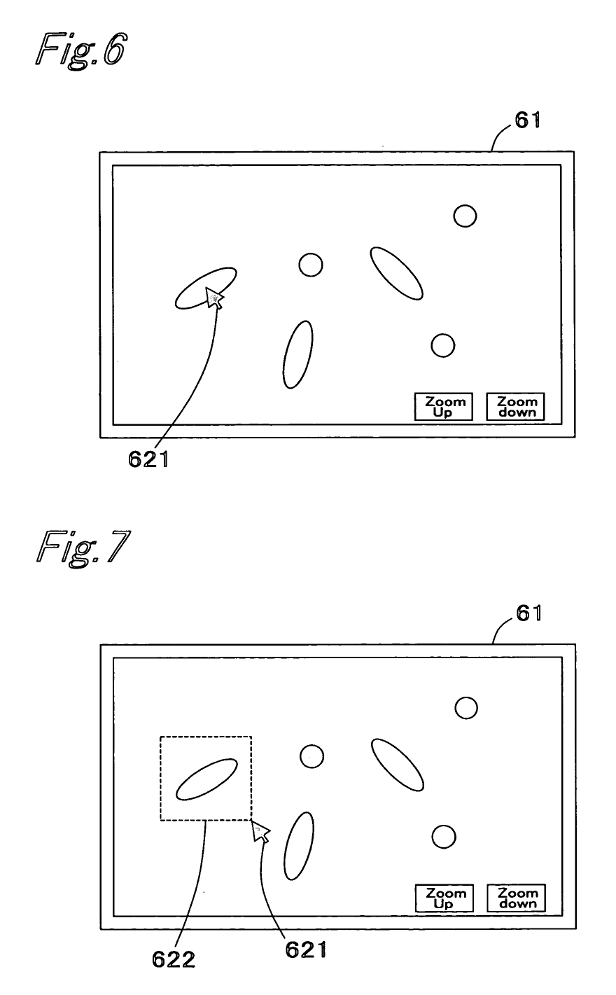

FIG. 6 represents a display (61) exhibiting the images obtained by the camera. The display (61) shows the image of the sample on the well (32) and a cursor (621) that moves in conjunction with the movement of the mouse device (62).

The display (61) shows at least a part of the observable area (341) of the object glass (34) shown in FIG. 5. The image on the display (61) can be either zoomed up or down as desired. In the example of FIG. 7, the display (61) has a zoom up button and a zoom down button on the lower right screen. Clicking on these buttons with the cursor (621) enables zooming up or down the image on the display (61).

The cursor (621) is moved to one cell by moving the mouse device (62). In order to specify the cell to be destroyed, the mouse device is clicked when the cursor points given place in the cell.

Then the computer (6) starts the binarization process from the clicked point to the periphery. This binarization process is completed when one isolated closed curve is identified. After that, the laser light is irradiated from the second laser light source (22) to the cells that have undergone the binarization process shown in FIG. 2. Thus the cells specified by the mouse device operation are destroyed.

FIG. 7 shows an example of cell destruction process using another method for specifying cells to be destroyed. In this method, processing area (622) is specified by the cursor (621) with dragging the mouse device (62). In FIG. 7, the processing area (622) is indicated with a dotted line. Then the binarization process is carried out within this processing area (622). The binarized cells are subsequently irradiated with the laser light from the second laser light source (22) as described referring to FIG. 2. Thus the cells within the area specified by the mouse device operation are destroyed.

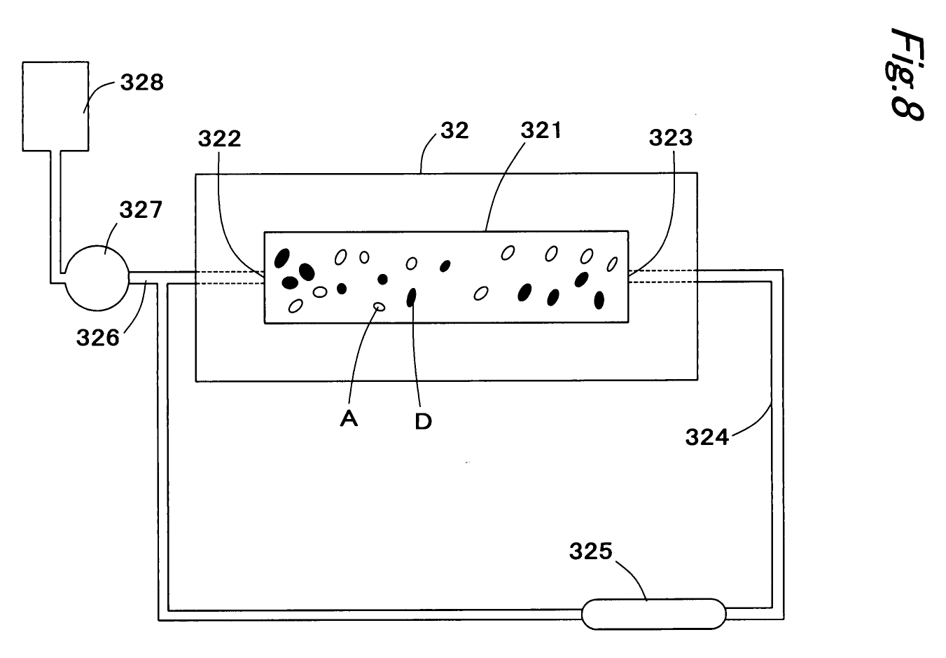

FIG. 8 shows one embodiment of the well (32).

The well (32) in FIG. 8 has a sample housing (321) with an inlet port (322) and an outlet port (323). The well (32) also has a circulation channel (324) between the inlet port (322) and the outlet port (323). In the middle of the circulation channel (324), a debris removal equipment (325) is deployed. The well (32) further comprises a branch (326) around the inlet port (322). The branch (326) connects to a pump (327) and a tank (328). The pump (327) provides normal saline solution to the inlet port (322) and the tank (328) stores the normal saline solution.

Note that, in the example of FIG. 8, the cells in the sample housing (321) have already undergone the cell destruction process. The destroyed cells (D) are black. The other cells (A) are white.

On running the normal saline solution into the inlet port (322) by rotating the pump (327), the sample in the sample housing (321) is flown toward the outlet port (323). The sample then flows down the circulation path (324) to the debris removal equipment (325). The debris removal equipment (325) captures the destroyed cells (D). As a debris removal equipment (325), any debris removal filters can be used. The cells (A) that are alive is carried back to the sample housing (321) through the debris removal equipment (325).

This circulation is not mandatory and sample that has passed through the debris removal equipment (325) may be introduced to other culturing wells.

Thus destroyed cells (D) and cells (A) that are alive can be collected separately.

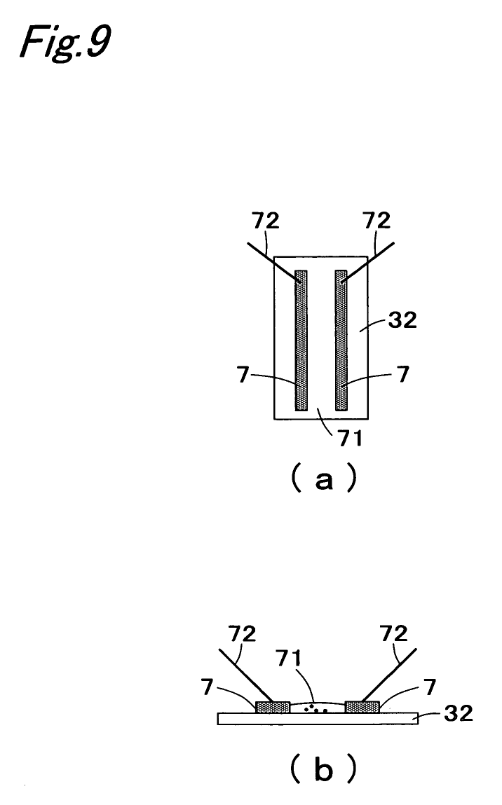

FIG. 9 shows another embodiment of the well (32). FIG. 9(a) is a top view of the well. FIG. 9 (b) is a cross-sectional view of the well. The well (32) in FIG. 9 is a glass product. The rectangular recess containing samples (321) is provided on the center of the well (32). The electrodes are placed along the opposite sides of the recess (321). A space between a pair of electrodes is defined as a slit (71). The electrodes (7), for example, consist of ormolu electrodes. The distance between electrodes (7) is not particularly restricted, however, it can be within 2 mm or be roughly 250 μm. In addition, the slit (71) might have a partition disposed at the right angles to the electrodes (7), so that the slit (71) can be divided into two spaces.

A suspension including cell samples is provided with the slit (71). The above-mentioned cell-destruction processing can be operated with cells in the suspension in the slit (71). Or the suspension containing only viable cells can be provided with the slit (71) by separating and removing the above-mentioned destroyed cells from the suspension after the cell-destruction processing.

The connectors (72) are connected to the electrodes (7). The electrodes (7) are electrically connected to a power source (not shown in the figure). Although the power source needs to apply at least an alternating voltage, it is more preferable that the power source can apply both alternating and direct voltages.

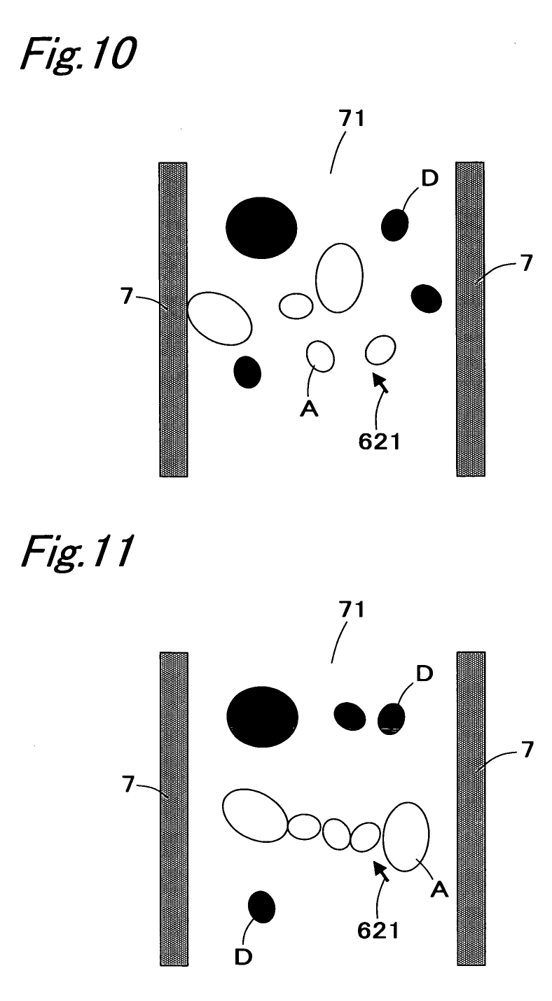

FIG. 10 describes the well (32) shown on the display (61). This represents the condition after the above-mentioned cell-destruction processing is operated for some of cells in a suspension. In FIG. 10, destroyed cells (D) are depicted as blackened circles, whereas viable cells (A) are depicted as whitened circles. The operation for arranging viable cells (A) to form a cell arrangement and the operation fusing cells in the cell arrangement will be described below.

The above-mentioned cell-destruction processing destroys unnecessary cells, thereby removing completely adverse effects attributed to incorporating unnecessary cells in the arrangement.

After the cell-destruction processing is completed, the cell arrangement scattering in the slit (71) does not have regularity at all. Under such circumstances, the viable cells (A) in a suspension are trapped with the IR laser of the first laser light source (21) directed into the slit (71) of the well (32). Driving the electric mirror unit (45) allows the cell arrangement to be approximated to the desired configuration.

Furthermore, it is preferable to use the cursor (621) to select a cell and to move the cell in conjunction with shifting the cursor (621) with monitoring the display (61).

In this phase of the process, it is not necessary that the cell arrangement after shifting is identical to the desired configuration. Thus, it is just required to be roughly approximated to the desired configuration. The subsequent condition due to this shift-operation is shown in FIG. 11.

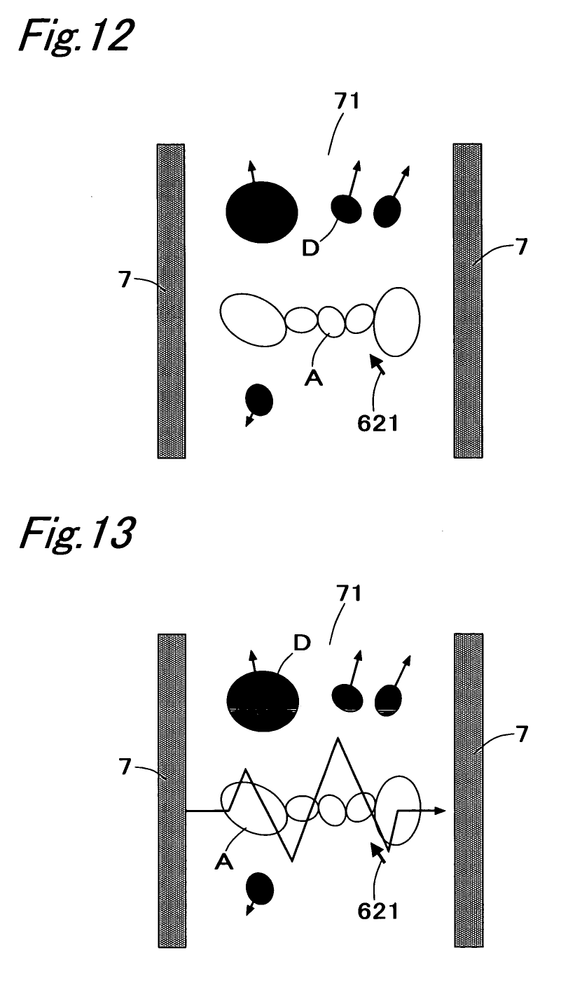

FIG. 12 describes the condition after an alternating voltage is applied to the electrodes (7). As shown in FIG. 11, after approximating the viable cells to the desired configuration, the electric field in a suspension disposed in the slit (71) is generated by applying an alternating voltage to the electrodes (7).

One of the conditions for an applied alternating voltage can be exemplified below: the voltage: ranging approximately from 6˜10 V, the frequency: around 2 MHz, and time of an applied alternating voltage: a few seconds˜around 20 seconds. It is worth noting that the condition for an applied voltage can be arbitrarily varied and optimized depending on types of cells and so forth.

Once applied an alternating voltage to the electrodes (7), cells (A) existing between electrodes commence moving so that they are aligned parallel to the electric field.

While forming the alignment, cells (A) move so that their trajectories become the shortest, shifting cells (A) to take the shortest distance virtually leads to the desired configuration because laser-trapping prior to applying an alternating voltage moves cells (A) where a cell contacts with the adjacent cell.

An alternating voltage also influences on destroyed cells (D), which may allow destroyed cells (D) to enter the configuration of viable cells (A). In this case, trapping destroyed cells (D) with the laser, these destroyed cells (D) can be moved so as to keep away from the cell configuration (the directions of arrows in FIG. 12).

Furthermore, a collapse of the configuration of a cell arrangement might result from a move of the viable cells (A) when applying an alternating voltage. In such a case, one of the cells in the approximate arrangement can be trapped with the laser to avoid the collapse. Hence, the laser-trapping operation may accurately form the desired cell arrangement at short times.

These laser-trapping operations can be conducted with moving IR lasers produced by the first light laser (21), as described above. After a certain period of time, cells stick to the well (32), which may make it difficult to move the cells by laser-trapping. However, the applied alternating voltage described above can tear off cells on the well (32), which facilitates the move by these laser-trapping operations.

FIG. 13 describes the process for fusing the viable cells (A) in the cell arrangement by applying a direct voltage to the electrodes (7) after arranging the viable cells (A) in the desired configuration. Adjoining viable cells (A) are fused due to the applied direct voltage to the electrodes (7). Thus, UV lasers produced by the second laser light source (22) can be used for this fusion. At this time, since cells in the arrangement can be placed closely, a conventional operation for adjoining cells by means of a multiple optical axis is no longer necessary. Consequently, quite a complicated optical system for introducing laser beams into the microscope (3) cannot be required.

The destroyed cells (D) are explained as unnecessary cells which should not be incorporated into the cell arrangement, but it is possible to treat the viable cells (A) as the unnecessary cells.

If the number of the viable cells (A) is greater than that required for making the cell arrangement in the suspension containing just the viable cells (A) after removing the destroyed cells (D) as described with FIG. 8, some of the viable cells (A) can be treated as the unnecessary cells.

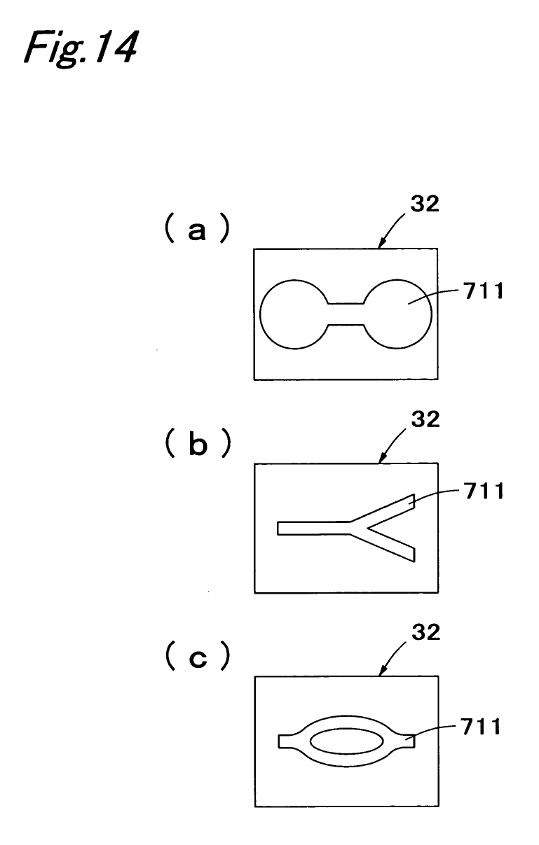

FIG. 14 represents another embodiment of the well (32). FIG. 14 (a) to (c) describe the wells (32) provided with grooves (711) in the slits (71) respectively. However, the shapes of grooves differ from each of them.

Furthermore, as shown in these figures, only slits (71) are extracted. However, as for the electrodes (7), the same structure shown in FIG. 9 can be employed.

The slits (71) of the wells (32) in FIG. 14(a) to (c) comprises grooves (711). Thus, the wells (32) with grooves (711) can be created by laminating flat surfaces on the basis of Stereolithography.

Thus, the grooves (711) can be shaped according to the desired cell arrangement (i.e. finished shape of the cell arrangement). The length, depth, and width of grooves (711) can be equivalent to or slightly larger than dimensions of the desired arrangement.

The groove in FIG. 14 (a) shapes two large circles connected by a rectilinear groove. The groove in FIG. 14(b) is shaped like “Y”. The groove in FIG. 14(c) is elliptical-shaped. However, as for the present invention, the grooves (711) in FIG. 14 are not limited to the shape. Hence, the shape can be varied depending on the desired cell arrangement.

A shift-operation for cells using the well (32) with grooves as indicated in FIG. 14. Thus, the well described below comprises the same groove (711) as FIG. 14 (a) is explained as below.

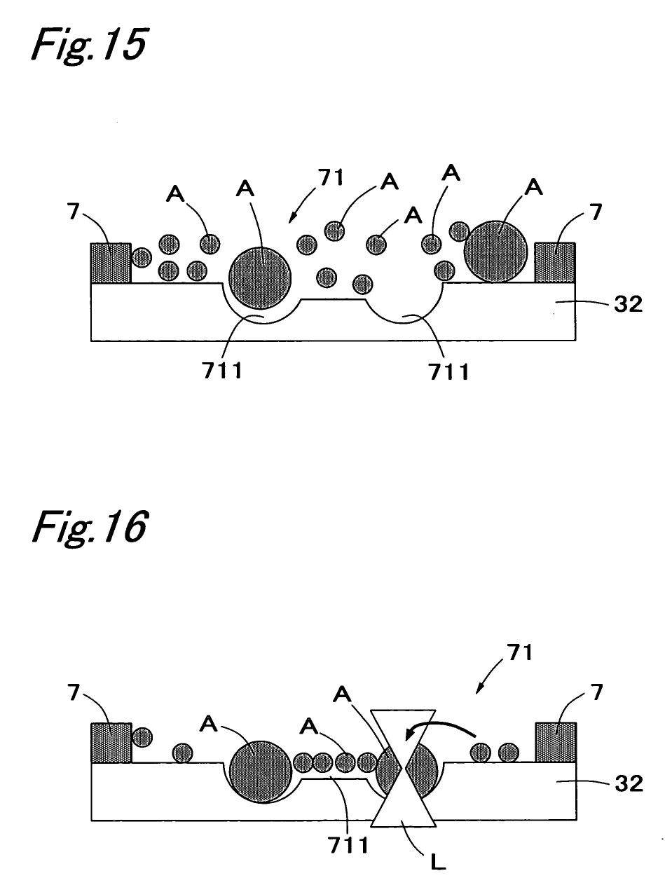

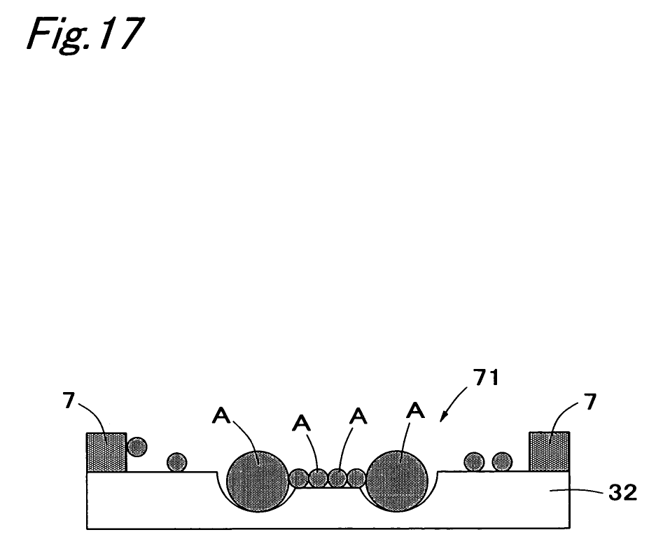

FIG. 15 to FIG. 17 show cross-sectional views of the well (32). FIG. 15 indicates that a suspension with cells is provided with the slits (71). FIG. 16 indicates that the cells in the suspension are moved by laser-trapping. FIG. 17 indicates that an alternating voltage is applied. Hence, cells incorporated into the cell arrangement are referred to a symbol (A).

As shown in FIG. 15, the cells in the suspension are irregularly dispersed either in a condition that a suspension is provided with the slit (71) or in a condition immediately after the cell-destruction processing is operated. As mentioned above, a laser light (L) of the first laser light source (21) traps a cell (A) in the suspension, which moves the cell (A). By this shift-operation, cells (A) to be incorporated into the cell arrangement are fallen to the groove (711), while unnecessary cells for the cell arrangement are moved far away from the groove (711) (See FIG. 16). Thus, after the cells (A) are deployed within the groove (711) and the cell arrangement in proximity to the desired cell configuration is formed, an alternating voltage is applied to the electrodes (7) (See FIG. 17). The applied alternating voltage allows cells (A) in the groove (711) to contact with each other, which makes it possible to obtain the desired cell configuration. Afterward, the cells in the cell arrangements are fused by exposure from the laser light to cells in the cell arrangement or by an applied direct voltage to the cell arrangement.

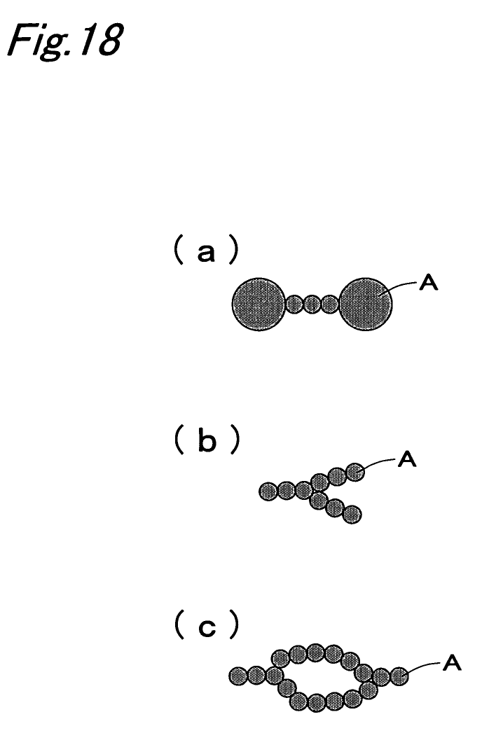

FIG. 18 shows cell arrangements provided with configurations of each groove of FIG. 14(a) to FIG. 14(c). FIG. 18(a) represents the cell arrangement shaped with the well (32) of FIG. 14(a). FIG. 18(b) represents the cell arrangement shaped with the well (32) of FIG. 14(b). FIG. 18(c) represents the cell arrangement shaped with the well (32) of FIG. 14(c).

In the case of use of the well (32) with a groove (711), an arbitrary cell arrangement can be easily obtained depending on the shape of the groove (711). Therefore, cell fusion is possible to occur with a wide variety of configurations. Furthermore, cells fused with a large number of cells at once can be obtained.

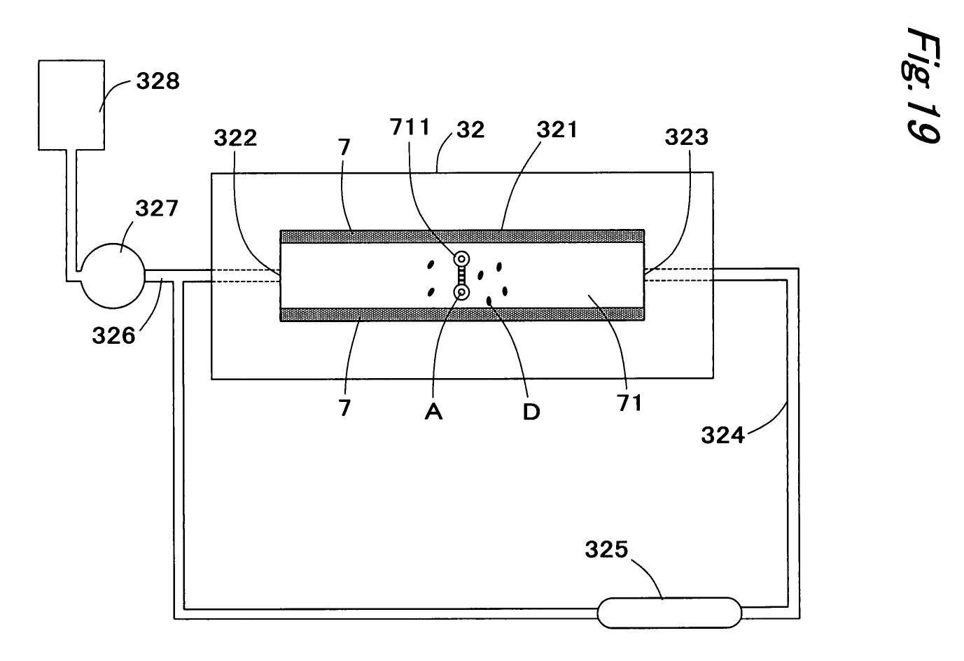

FIG. 19 describes the cell handling system having the well (32) with electrodes (7) and the above-mentioned groove (711). As for FIG. 19, viable cells (A) are necessary, whereas destroyed cells (D) are not necessary.

First of all, the viable cells (A) are trapped by the laser. Afterward, these viable cells (A) are placed in the groove (711). Then, destroyed cells (D) are moved to the downstream of the slit (71) (i.e. the side of the outlet (323)). This cell-shift operation is conducted by applying an alternating voltage to electrodes (7), as mentioned above.

After moving all of the destroyed cells (D) in the downstream of the slit (71), physiological saline is provided for the slit (711) from the inlet (322). At this time, the flow from the inlet (322) is set so that the viable cells (A) in the groove (711) are not overflowed from it. Besides, during this operation, an alternating voltage can be applied in order to facilitate the flow of destroyed cells (D).

While the viable cells (A) are stopped by the groove (711), the destroyed cells (D) flow to the downstream of the groove (711), and then are captured by the debris removal equipment (325). Thus, this operation enables to efficiently separate and recollect cells as either necessary or unnecessary ones.

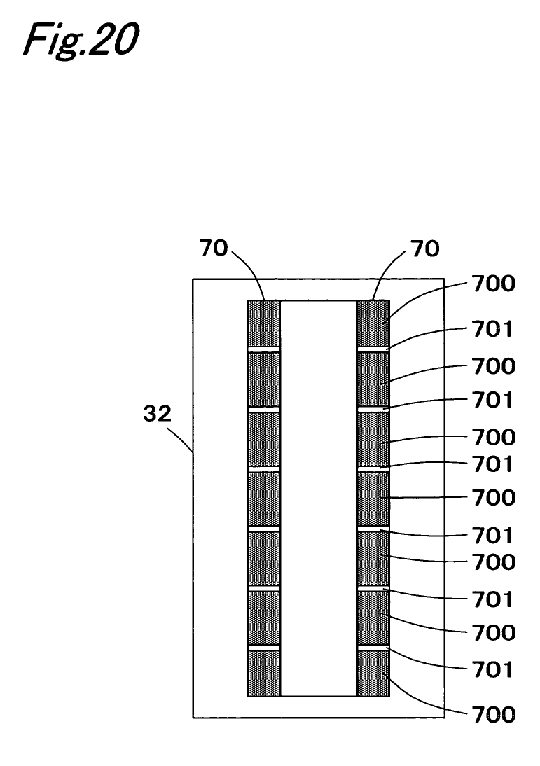

FIG. 20 represents another configuration of the well (32). On the above-mentioned example, electrodes (7) on the well (32) are a pair of platy or virgulate electrodes. However, segmented electrodes (70) can be used as shown in FIG. 20. Insulators (701) are placed between electrode segments (700). Electrodes segments themselves (700) are electrically isolated. In addition, not shown in the figure, each electrode segment (700) is independently connected to the power source.

Thus, the segmented electrodes (70) applies an alternating voltage only to the part as one desires that the cell peel-off effect is locationally achieved, which makes it possible to conduct more complex cell shift operations.

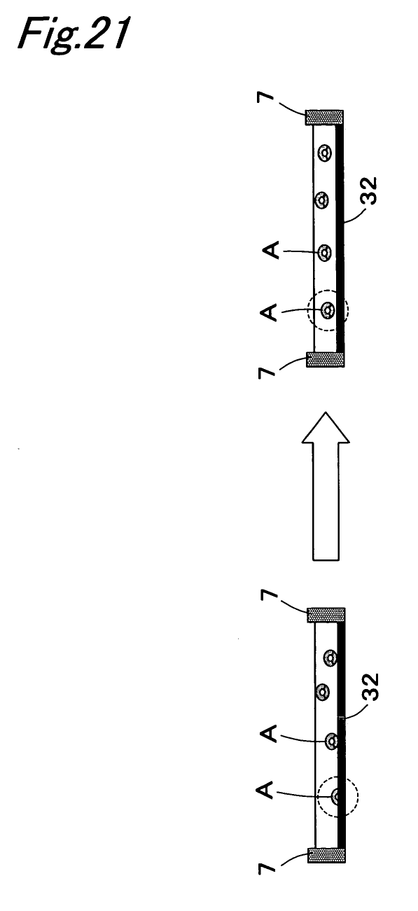

FIG. 21 describes the cell-shift operation by laser-trapping while an alternating voltage is applied. As for the above-mentioned example, the cell-shift operation by laser-trapping before applying the alternating voltage. However, laser-trapping can be carried out while applying the alternating voltage.

An example shown in FIG. 21 illustrates that one of the cells in a suspension disposed in the slit (71) between electrodes (7) adheres to the bottom of the well (32) (i.e. the cell surrounded by dotted lines in FIG. 21). Thus, under such conditions, it is difficult to move the cell adhering to the well bottom by laser trapping because of insufficient trapping force.

In this case, an alternating voltage can be applied to electrodes (7) for a few seconds (the period of time that cells are slightly migrated as well as a pearl chain is not formed). As a result, the cell that is on the bottom of the well, or that is about to adhere to the bottom can be taken off the surface of the well (32). Thus, the cell on the bottom of the well (32) floats in a suspension, which dramatically facilitates the shift by laser-trapping. Therefore, the present invention widely improves efficiency of laser manipulation, and it allows the cell operation to be conducted without losing cells' freshness in the case of treating viable cells. In addition, this invention is very useful for conducting the cell operation that only specific types of cells selectively picked up from suspension containing several types of cell undergo some treatments (e.g. fusion and culture) by completely destroying the other types of cells.

The present invention is applied to a cell-operating device and cell operating method that facilitate the complete destruction of unnecessary cells and the operation of necessary cells in a suspension.

1. A cell-operating device comprising:
a laser light source;
a microscope to observe cell sample fluorescently labeled;
a camera to take the images of the cell sample that is observed with said microscope;
a computer to capture the digital image data obtained by said camera; and
a group of mirrors comprising electronic control mirrors of which optical axis can be adjusted so that the laser light irradiated from said laser light source can be guided in to said microscope, and
wherein said computer includes:
a binarization program for binarizing said digital image data by identifying whether each pixel of said digital image data captures a part of said cell sample according to the fluorescence luminance of each pixel;
an identification program for identifying the boundaries between adjacent different values in the binarized digital image and joins the boundaries into an isolated closed curve;
a data processing program for processing said binarized digital data so that in all the area within said isolated closed curve a cell exists;
a calculation program to divide said binarized digital image data, after running said data processing program, into sections with a predefined size and identify the center of gravity coordinate of each section; and
an adjustment program to adjust the optical axis of said electronic control mirrors so that said laser irradiates said center of gravity coordinate; and
wherein laser light from said laser light source irradiates said center of gravity coordinate.

2. A cell-operating device of claim 1, wherein said computer provides a filtering program comprising the steps of, calculating the square measure within said isolated closed curve just after running said data processing program by counting the number of pixels within said isolated closed curve of said image data, and excluding areas smaller than predefined size from destruction target.

3. A cell-operating device of claim 1, wherein the size of said sections is defined as a rectangular area that is larger than the rectangular area that the spot of said laser light circumscribes and that is smaller than the rectangular area that the spot of said laser light inscribes.

4. A cell-operating device of claim 1, comprising a display that shows at least a part of the image from said camera.

5. A cell-operating device of claim 4, further comprising a mouse device, wherein said display can show a cursor that moves in conjunction with the movement of said mouse device and the isolated closed curve for the cells pointed by said cursor is identified by said identification program.

6. A cell-operating device of claim 5, further comprising a mouse device that can point an area on said display, wherein said pointed area binarized by the binarization program.

7. A cell-operating device of claim 1, further comprising a well on which said cell sample is placed and a debris removal equipment that connects to said well.

8. A cell-operating device of clam 7, comprising a circulation channel that connects said debris removal equipment to said well.

9. A cell-operating device of claim 1 comprising:
a pair of electrodes attached on said well so that a slit in which the cell sample is placed is formed between said electrodes;
an electric source that can generate alternating voltage between said pair of electrodes; and
wherein said laser light source can trap cells.

10. A cell-operating device of claim 9, wherein a groove is formed in said slit.

11. A cell-operating device of claim 1, further comprising a well on which the cell sample is placed and an electric stage that moves said well in two directions on a horizontal plane.
